(19) United States
(12) Patent Application Publication
(10) Pub. No.: US 2013/0220337 A1
(43) Pub. Date: Aug. 29, 2013

(54) DEVICES AND METHODS FOR INTRAVAGINAL DELIVERY OF DRUGS AND OTHER SUBSTANCES

(71) Applicants: Patrick F. Kiser, Salt Lake City, UT (US); Vlad G. O'mel, Midvale, UT (US); Leland J. Prather, Salt Lake City, UT (US); Festo Damian, Etobicoke (CA)

(72) Inventors: Patrick F. Kiser, Salt Lake City, UT (US); Vlad G. O'mel, Midvale, UT (US); Leland J. Prather, Salt Lake City, UT (US); Festo Damian, Etobicoke (CA)

(21) Appl. No.: 13/818,067

(22) PCT Filed: Aug. 19, 2011
(86) PCT No.: PCT/US11/48453

§ 371 Date: May 6, 2013

Related U.S. Application Data
(60) Provisional application No. 61/375,671, filed on Aug. 20, 2010.

(51) Int. Cl. A61M 31/00; A61F 6/06

(52) U.S. Cl. CPC A61M 31/002; A61F 6/06
USPC 128/832; 604/285; 53/473; 53/438

(57) ABSTRACT

The present technology provides intravaginal devices designed to intravaginally deliver difficult to formulate water-soluble molecules and macromolecules for a sustained period of time. The devices include a chamber having at least one orifice and containing a sustained-release formulation. Such formulations include a water-swellable polymer and an intravaginally administrable substance. Methods of making and using the devices are also provided.

### FIELD

The present technology relates to devices and methods for intravaginal delivery of drugs and other substances, including, e.g., the sustained administration of water-soluble drugs and macromolecules by an intravaginal device.

### BACKGROUND

A limited range of drugs and other substances, may be delivered intravaginally using devices such as intravaginal rings. For example, several commercially available intravaginal rings provide controlled and sustained delivery of steroid molecules over several days, including ESTRING, FEMRING, and NUVARING. Such intravaginal rings are reservoir type delivery systems, and comprise a core of a polymeric material loaded with the substance to be delivered. Accordingly, loading and release of substances from such delivery system are dependent upon the solubility and diffusion of the substance in a polymeric matrix. To date, the polymeric materials used in the construction of commercial intravaginal rings have been limited to hydrophobic polymers; consequently, current rings are limited to delivery of hydrophobic substances. Moreover, diffusion of substances through the polymeric matrix sharply depends on the molecular mass of the substance and porosity of the matrix. Hence, another disadvantage of reservoir intravaginal rings constructed from the hydrophobic polymeric materials is that they are not applicable for delivery of macromolecules.

Various strategies to increase the range of substances which may be delivered intravaginally have been proposed. In one method, the loading of the substance is increased to, e.g., greater than 20% w/w of the intravaginal ring. While such high substance concentrations can provide improved flux of the loaded substances from outer layers, they waste any substance, e.g., a drug, located in the core of the device. In addition, high concentrations of drugs or other substances in the polymer matrix can adversely affect required mechanical properties of such rings and therefore still limit the range of substances which may be delivered.

In another strategy, water-soluble release enhancers may be incorporated into the polymer matrix of the intravaginal ring. Water/fluid uptake into matrix results to release of the enhancers and thus increases matrix porosity. However, high loadings, e.g., greater than 15%, of the water-soluble release enhancers are required to significantly enhance the release rate of the drug or other substances. Such high loadings are problematic for manufacturing the rings and can result in excessive swelling and expansion of the device during use such that its original shape and size are no longer maintained.

In another strategy, a polymeric insert containing the drug or other substance is sealed in a bore extending into the ring from the ring surface. Because the insert is completely surrounded by the non-medicated polymeric membrane, the polymer of the ring controls the rate of substance release. This design is used to produce zero-order or near zero-order release (constant daily release) via a permeation-controlled mechanism (molecular dissolution and subsequent diffusion). In this strategy, the range of substances that may be delivered is still constrained by the compatibility of the substances with the ring polymeric matrix and the limited porosity of the matrix.

### SUMMARY

The present technology provides, devices and methods for intravaginal delivery of sustained release formulations of various substances. The devices are especially suited for delivery of hard-to-formulate water soluble drugs and macromolecules, though other drugs or substances may also be used. The devices include a chamber having at least one orifice. The chamber contains a sustained-release formulation, wherein the formulation includes a water-swellable polymer and an intravaginally administrable substance. Because the orifice allows water to directly contact the sustained release formulation, causing it to swell and be expelled from the chamber in a controlled fashion, the present devices overcome various constraints imposed by traditional substance-impregnated polymer intravaginal devices. First, the substances do not have to diffuse through a hydrophobic polymer into the vaginal environment, the present devices eliminate the problems arising from the incompatibility of certain types of substances with the polymer matrices traditionally used to intravaginally deliver such substances. Moreover, the present devices offer exquisite control of substance release rate based on the selection of water-swellable polymer, mixture with excipients, compression of the water-swellable polymer and the size and number of the orifice.

### BRIEF DESCRIPTION OF THE DRAWINGS

FIGS. 1 and 2 each show a schematic of an illustrative embodiment of a substance release chamber (pod) according to the present technology.

FIG. 3 shows an illustrative embodiment of a intravaginal ring of the present technology, showing bores for the substance release chambers.

FIG. 4 shows an illustrative embodiment of the present technology in which an intravaginal ring is loaded with two substance release chambers.

FIG. 5 shows a typical calibration curve for quantification of carrageenan release into VFS.

FIG. 6 shows the cumulative release of iota-carrageenan type V (Sigma) from a substance release chamber (i.e., pod) of Example 1. The plot is the average release obtained from three pellets. The error bars show the standard deviation.

FIG. 7 shows the cumulative release of iota-carrageenan (Gelcarin PC379, FMS Biopolymer) from an illustrative embodiment of a device of the present technology. The plot is the average release obtained from three pellets. The error bars show the standard deviation.

FIG. 8 shows the effect of the cationic excipient lysine on the rate of carrageenan release. The plot is the average release obtained from three pellets. The error bars show the standard deviation.

FIG. 9 shows the release BSA-FITC from an iota-carrageenan matrix. Content of BSA-FITC is 250 μg per 100 mg of carrageenan. The plot is the average release obtained from three pellets. The error bars show the standard deviation.

FIG. 10 shows the effect of compression force on release of iota-carrageenan from an illustrative embodiment of a device of the present technology. The plot is the average release obtained from three pellets. The error bars show the standard deviation.

FIG. 11 shows the effect of orifice size on the rate of carrageenan release from an illustrative embodiment of a pod of the present technology. The plot is the average release obtained from three pellets. The error bars show the standard deviation.

FIG. 12 shows the effect of swelling intensity of pellet matrix upon hydration on rate of carrageenan release from an illustrative embodiment of a device of the present technology as show by adding 20 w % of carbopol to iota-carrageenan. The plot is the average release obtained from three pellets. The error bars show the standard deviation.

FIG. 13 shows release of fluorescein as a model of a low molecular weight water soluble drug from an illustrative embodiment of a pod of the present technology containing a iota-carrageenan matrix. The error bars show the standard deviation. The plot is the average release obtained from three pellets.

FIG. 14 shows the effect of 20 wt % ethyl cellulose on release of carrageenan from an illustrative embodiment of a pod of the present technology. The error bars show the standard deviation. The plot is the average release obtained from three pellets.

FIG. 15 is a graph showing the cumulative release of TAMRA-insulin from an illustrative embodiment of a device of the present technology using three different polymer matrices: iota-carrageenan, hydroxy ethyl cellulose (HEC) and hydroxy propyl cellulose (HPC). Each pod contains two 1.5 mm diameter release orifices with a 100 mg pellet of 1.0 wt % TAMRA-insulin in iota-carrageenan, HEC and HPC. Release conditions: 37° C., 80 rpm in 25 mM acetate buffer, pH 4.2. (N=3, Mean±S.D.).

FIG. 16 is a graph showing the cumulative release of TAMRA-insulin from an HPC matrix in an illustrative embodiment of a device of the present technology using three different molecular weight HPCs. Each pod contains two 1.5 mm diameter release orifices with a 100 mg pellet of 1.0 wt % TAMRA-insulin in Klucel HPC, LF, JF or GF Pharm. Release conditions: 37° C., 80 rpm in 25 mM acetate buffer, pH 4.2. (N=3, Mean±S.D.).

FIG. 17 is a graph showing daily release of rhodamine-B dextran from an HPC matrix in an illustrative embodiment of a device of the present technology. Each pod contains two 1.5 mm diameter release orifices with a 100 mg pellet of 1.0 wt % rhodamine-B dextran in Klucel HPC GF Pharm. Release conditions: 37° C., 80 rpm in 25 mM acetate buffer, pH 4.2. (N=3, Mean±S.D.).

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here.

The present technology provides devices and methods for sustained release of substances, such as drugs, intravaginally. In particular, intravaginal devices of the present technology extend the range of drug types that may be delivered intravaginally to water-soluble drugs and macromolecules. Thus, in one aspect, the present technology provides intravaginal devices which include a chamber having at least one orifice (also referred to as a pod herein). The chamber contains a sustained-release formulation, wherein the formulation includes a water-swellable polymer and an intravaginally administrable substance. By “sustained release formulation” is meant a formulation of the intravaginally administrable substance that is released over the course of a period of one or more hours. In some embodiments, the sustained release of the substance occurs over the course of at least 2, at least 4, at least 6, at least 8, at least 12, at least 18 or at least 24 hours. In other embodiments, the sustained release of the substance occurs over the course of at least 1, at least 2, at least 3, at least 4, at least 5, at least 6, at least 7, at least 10, at least 20 or at least 30 days or over the course of at least 1, at least 2, at least 3 or at least 4 weeks, or even over the course of 1, 2 or 3 months.

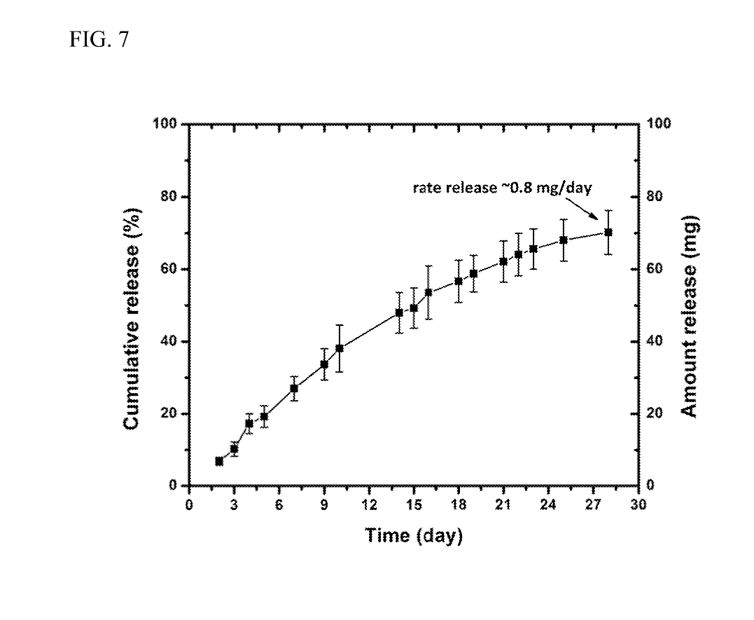

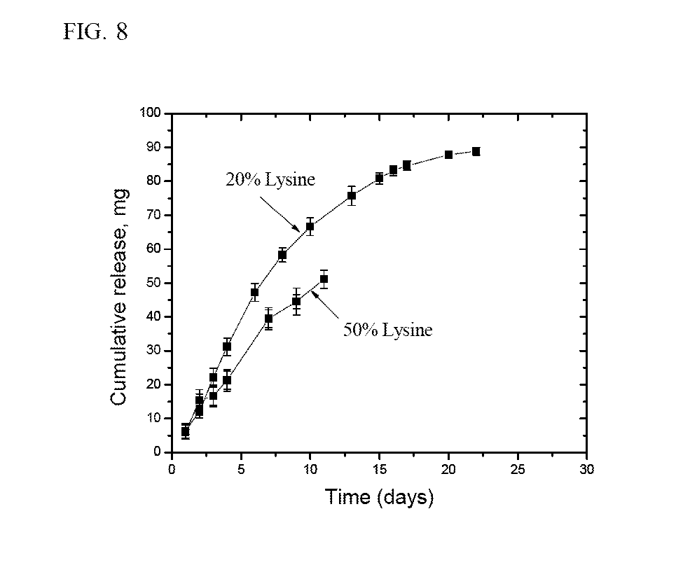

Water-swellable polymers that may be used in the intravaginal devices include any biocompatible polymers that swell on contact with water, e.g., anionic or cationic polymers (including zwitterionic) or nonionic water soluble polymers. Such polymers include but are not limited to carrageenan, polyacrylic acid, polyvinyl pyrrolidone, polyvinyl alcohol, or hydroxyl ethylcellulose with molecular weights between 10,000 and 1,000,000. Examples of molecular weights of such polymers include 10,000, 20,000, 30,000, 40,000, 50,000, 75,000, 100,000, 150,000, 200,000, 300,000, 500,000, 750,000, 1,000,000 and ranges between and including any two of these values. The rate of release of a substance from the present intravaginal devices may be controlled by controlling the rate of swelling of the water-swellable polymer. One way to control release rates is therefore through the selection of water-swellable polymers having different rates of swelling Thus, for example, lambda carrageenan, iota carrageenan, kappa carrageenan or a mixture of any two or more thereof may be used as the water-swellable polymer to adjust release rates (see, e.g., FIGS. 7 and 8).

The release rate may further be controlled by the addition of excipients to the sustained release formulation. Depending on the effect desired, excipients may increase, reduce or leave the rate of release of the formulation the same. Suitable excipients include but are not limited to those selected from the group consisting of cellulose, xanthan gum, amino acids, and glucosamine. For example, the excipient may be selected from the group consisting of lysine, histidine, or arginine. In some embodiments, the excipient is ethyl cellulose. Cationic excipients such as lysine or arginine can be added to slow the release rate (see, e.g., FIG. 8). In some embodiments, about 0.5 wt % to about 60 wt % (based on the total weight of the sustained release formulation) of a cationic excipient is added to the sustained release formulation. In other embodiments the cationic excipient ranges from about 1 wt %, about 5 wt % or from about 10 wt % to about 50 wt %, from about 20 wt % to about 50 wt % or from about 20 wt % to about 40 wt % of the sustained release formulation.

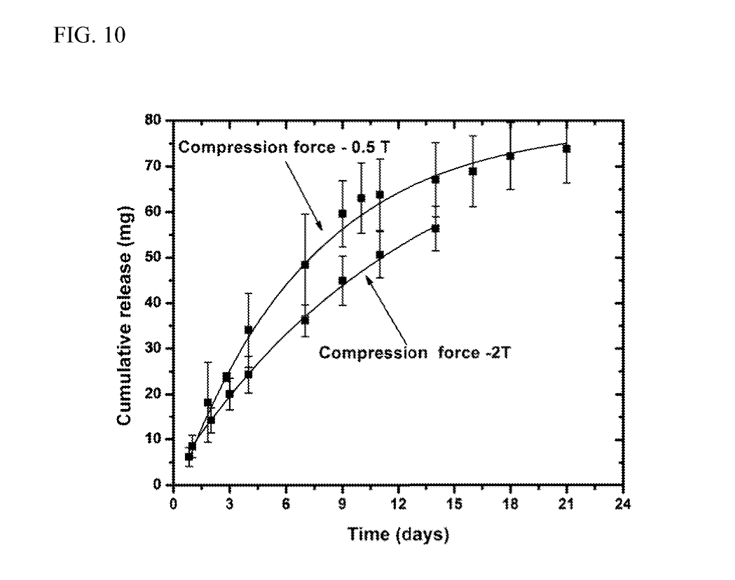

The release rate may be further controlled by compression of the sustained release formulation into a pellet. As shown in FIG. 10, higher compression pressures lead to lower release rates. Thus, in some embodiments, the pellet may be compressed with a force from about 0.1 tons/inch (T) to about 10 T, or from about 0.5 T to about 1 T, about 2 T, about 3 T, about 4 T, about 5 T, about 6 T, about 8 T and ranges between and including any two of these values.

In intravaginal devices of the present technology, the sustained release formulation is contained in a chamber with at least one orifice (i.e., a pod). By “orifice” is meant a hole through which the sustained release formulation may directly pass into the vaginal environment without having to diffuse through a polymeric barrier. The chamber is sealed except for the orifice(s) through which the formulation passes. The orifice must be large enough to allow a sufficient amount of water to pass into the chamber and swell the water-swellable polymer. The orifice must also be sufficiently small such that the formulation is released over the desired time period of, e.g., days, weeks or months. The orifice may have any suitable shape, including but not limited to round, square or polygonal. In an illustrative embodiment, the chamber has a single orifice with a diameter of about 0.1 mm to about 4 mm. In other embodiments, the orifice has two, three, four, five or more orifices. In some embodiments, the orifice(s) have a diameter of about 0.5 mm to about 4 mm, about 1 to about 4 mm, about 1 to about 3 mm or about 1 to about 2 mm in diameter.

The size and shape of pods of the present technology may vary but are typically designed to hold from 50 to 500 mg of the sustained release formulation. In some embodiments the pods are cylindrical. Such pods may have a diameter ranging from about 3 to about 15 mm and a height ranging from about 1 to about 10 mm. In some embodiments the diameter ranges from about 5 to about 10 mm and the height ranges from about 2 to about 7 mm. In another embodiment the pods are solid objects made with the same curvature of the intravaginal device. In another embodiment the pod is shaped like a tampon.

The materials the pods are made out of may vary but medically acceptable polymers include polypropylene, nylon, polyurethane, polystyrene, polycarbonate and acrylonitrile butadiene styrene (ABS). An alternative material includes medically acceptable metals, e.g. stainless steel.

A variety of intravaginal devices may be fitted with a pod as described herein, including but not limited to an intravaginal ring, tampon or pessary. The intravaginal device may include one or two or more chambers, each including an orifice and each containing a sustained-release formulation. Where the device includes two or more such chambers (e.g., two to three, four, five or six), each may include the same sustained release formulation or one or more different sustained release formulations. In some embodiments, the intravaginal devices include two or three chambers, each including an orifice and containing a sustained-release formulation.

In some embodiments the intravaginal device is an intravaginal ring. Such rings may be made from elastomers that are either thermosets or thermoplastics. Thermoplastics include but are not limited to polyurethanes and ethylene vinyl acetate. Thermosets include but are not limited to the class of silicone polymers. Intravaginal rings may also be made from a mixture of any two or more polymers. The intravaginal ring may include a second intravaginally administrable substance the same or different from the first such substance. The second intravaginally administrable substance may be, e.g., loaded into a second chamber having at least one orifice and containing a sustained-release formulation comprising a second water swellable polymer (or into two or more additional such chambers).

Alternatively, the second intravaginally administrable substance may be loaded into the ring itself. In this case the substance may reside in one or more segments of the ring having a rate controlling sheath surrounding the core of the device (see, e.g., van Laarhoven, H., et al. (2004) Pharm Res 21, 1811-1817; van Laarhoven, J. A. (2005) Physical-Chemical Aspects of a Coaxial Sustained Release Device Based on Poly-EVA. in Pharmaceutics, University of Utrecht; van Laarhoven, J. A., et al. (2002) J Control Release 82, 309-317; van Laarhoven, J. A., et al. (2002) Int J Pharm 232, 163-173). Such sheaths are constructed from a polymeric membrane known to control the rate of diffusion of substances, e.g., ethylene vinyl acetate copolymers can be used to coat the drug loaded core to control the rate of release of the drug. Other polymers like crystalline polyurethanes can also be used as rate controlling sheaths. Alternatively silicones can be used both as the core and sheath materials such as in the well know estrogen replacement intravaginal rings: ESTRING and FEMRING.

In some embodiments of the present devices, the sustained release formulation is formulated to release the water-swellable polymer in vivo and/or in the presence intravaginal fluid simulant for at least 7 days, at least 14 days, or at least 30 days.

The sustained release formulations of the present technology may be formulated with a variety of intravaginally administrable substances. Such substances are compatible with the vaginal environment and include biologically active substances, such as vitamins and drugs, as well as biologically inert substances such as certain excipients. The drugs may be any which are locally useful in the vagina and/or are capable of crossing the vaginal membranes to exert systemic effects in a subject. The present devices are especially useful for delivering water soluble drugs (e.g., those having a solubility in water of at least 0.1 mg/mL at physiological pH and temperature) or macromolecules. In some embodiments, the water soluble drugs have a solubility in water of at least 0.2, at least 0.5, at least 1, at least 2, at least 5 or at least 10 mg/mL. Excipients may be formulated with the drugs or other substances or may be formulated alone and include, but not limited to, those described above as well as amino acids, disintegrants, lubricants, carriers, surfactants, fragrances, and the like.

Intravaginally administrable substances include intravaginally administrable drugs such as cervical anesthetics, contraceptives, antiendometriosis drugs, estrogen receptor modulators, preterm labor drugs, overactive bladder drugs, morning sickness drugs, osteoporosis drugs, antimicrobials, vaccines and the like. Thus, useful intravaginally administrable substances include but are not limited to cervical anesthetics, such as lidocaine, contraceptives such 17a-ethinyl-levonogestrel-17b-hydroxy-estra-4,9,11-trien-3-one, estradiol, etonogestrel, levonorgestrel, medroxyprogesterone acetate, nestorone, norethindrone, progesterone, estrogen receptor modulators such as RU-486, anti-endometriosis drugs such as Terbutaline, antivirals such as, acyclovir, and ganciclovir, blood flow increasing drugs like Sildenafil, labor inducing drugs like misoprostol, preterm labor drugs like indomethacin, overactive bladder drugs like Oxybutynin, morning sickness drugs such as Bromocriptine, osteoporosis drugs like human parathyroid hormone, drugs and/or substances for vaginal dryness such as glycerol and/or other lubricating or hydrating substances, pseudovirions such as HPV, HIV and HSV psuedovirions and antigens and/or other immunogens and combinations of any two or more thereof.

Intravaginally administrable substances may include antimicrobials such as antiviral, antifungal or antibacterial drugs. For example the drug can be an anti-human papilloma agent selected from the group consisting of sulfated polysaccharides or polyamidopyrroles. In some embodiments, lambda carrageenan, kappa carrageenan, iota carrageenan, a polypyrrole and a combination of any two or more thereof can be used. In another example, the drug can be an anti-HIV agent selected from the group consisting of non-nucleoside reverse transcriptase inhibitors, nucleoside reverse transcriptase inhibitors, integrase inhibitors, protease inhibitors and HIV entry inhibitors. In some embodiments, the non-nucleoside reverse transcriptase inhibitor is dapivirine and the nucleoside reverse transcriptase inhibitor is Tenofovir. In other embodiments, the substance is selected from 1-(cyclopent-3-enylmethyl)-6-(3,5-dimethylbenzoyl)-5-ethylpyrimidine-2,4(1H,3H)-dione, 1-(cyclopentenylmethyl)-6-(3,5-dimethylbenzoyl)-5-isopropylpyrimidine-2,4(1H,3H)-dione, 1-(cyclopent-3-enylmethyl)-6-(3,5-dimethylbenzoyl)-5-isopropylpyrimidine-2,4(1H,3H)-dione, 1-(cyclopropylmethyl)-6-(3,5-dimethylbenzoyl)-5-isopropylpyrimidine-2,4(1H,3H)-dione, 1-(4-benzoyl-2,2-dimethylpiperazin-1-yl)-2-(3H-pyrrolo[2,3-b]pyridin-3-yl)ethane-1,2-dione, or 19-norethindrone, AMD-3100, BMS-806 carrageenan, CD4-IgG2, cellulose acetate phthalate, cellulose sulfate, dapiravine, Dextrin-2-sulphate, efavirenz, etravirine (TMC-125), mAb 2G12, mAb b12, Merck 167, nonoxynol-9, plant lectins, poly naphthalene sulfate, poly sulpho-styrene, PRO2000, PSC-Rantes, Rilpivirine (TMC-278), Tenofovir, TMC-125, UC-781, efavirenz, viramune, etravirine (TMC-125), rilpivirine (TMC-278), tenofovir disoproxil fumarate, MIV170, MIV150, and PIE-12 trimer.

Macromolecules that may be delivered by the present devices include sulfated polysaccharides (e.g., carrageenan such as lambda carrageenan, iota carrageenan, and/or kappa carrageenan), proteins (e.g., insulin, HIV-1 envelope proteins, HSV envelope proteins, and therapeutic antibodies), polyacrylic acid, and carbopol, as well as polypyrroles (e.g., those described in K. S. Crowley et al./Bioorg. Med. Chem. Lett. 13 (2003) 1565-1570). In some embodiments, the water-swellable polymer and the intravaginally administrable substance are the same, such as, e.g., a carrageenan or a mixture of carrageenans.

In some embodiments, the intravaginally administrable substance is a sulfated polysaccharide and the second intravaginally administrable substance is selected from an anti-viral or a contraceptive. For example, the intravaginally administrable substance may be carrageenan, and the second intravaginally administrable substance may be a contraceptive loaded into one or more segments of the intravaginal ring which are surrounded by a sheath allowing for controlled release of the contraceptive.

In another aspect, the present technology provides methods of making the present devices. The methods include loading a sustained-release formulation into a chamber of an intravaginal device wherein the chamber has at least one orifice and wherein the sustained-release formulation includes a water-swellable polymer and an intravaginally administrable substance (such as an intravaginally administrable drug). The methods may include compressing particles of the water-swellable polymer and the substance, optionally mixed with one or more excipients as described herein, into a pellet before loading the pellet into the chamber of the intravaginal device. The particles of water swellable polymer may be put through a sieve having a mesh size of from about 200 μM to about 80 μM prior to compression. The particles may be compressed at a pressure of about 0.5 T to about 6 T (or other pressures described herein) to form the pellet. The water-swellable polymer used in the present methods include any such polymers described herein, such as carrageenan.

In another aspect, there are provided methods of administering a sustained release formulation using the present devices. The methods include releasing a substance from an intravaginal device comprising a chamber having at least one orifice and containing a sustained-release formulation, wherein the formulation comprises a water-swellable polymer and an intravaginally administrable substance. Any of the water-swellable polymers and intravaginally administrable substances as described herein may be used in the present methods.

### EXAMPLES

### Example 1

### Pod Construction

The pods were constructed by two methods.

Method 1A.

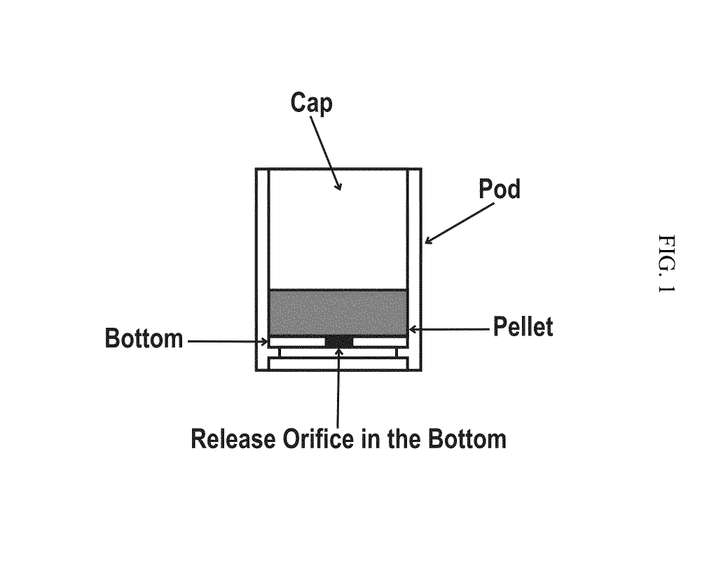

In one method, a 200 ul pipette tip was used to fabricate the pod. A schematic of this method is pictured as FIG. 1. The bottom of the pod was fabricated from a 0.5 mm thick polypropylene sheet. One or more release orifices of various diameters were drilled into the bottom of each pod. The bottom was tightly fit into the pod. The pellet was placed on the bottom and capped (sealed) with melted polystyrene. The completed pod is approximately 9 mm in diameter and 6 mm high, and holds a compressed pellet of from 50-400 mg of a formulation that includes a water-swellable polymer.

Method 1B.

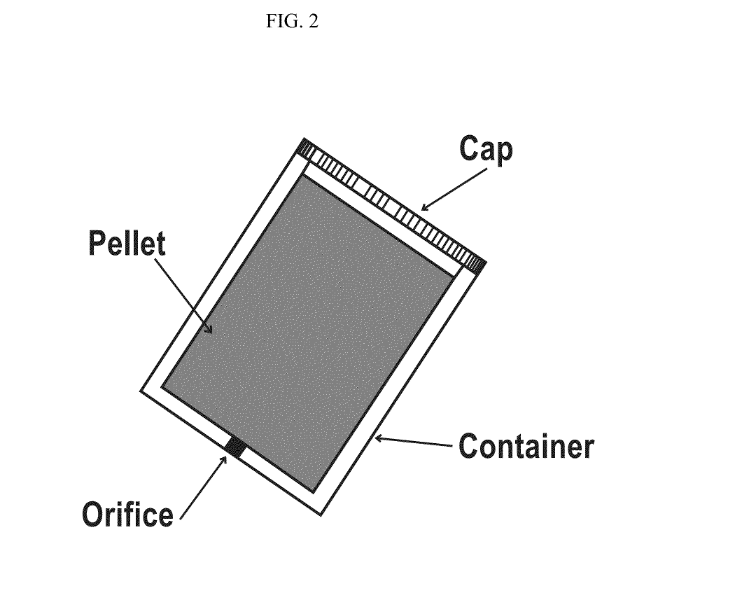

In another method, a prototype for industrial fabrication was produced by injection molding using any suitable biocompatible polymer such as polyurethane, polystyrene or silicone. The chamber of the pod with an orifice in the bottom was fabricated by 3D printing. The pellet was placed in container and sealed with cap of the same material. The cap was secured using an epoxy adhesive or cyanoacrylate adhesive. A schematic of the pod produced by this method is pictured on FIG. 2. The completed pod is approximately 7.3 mm in diameter and 5 mm high, and holds a compressed pellet of from 50-400 mg of a substance formulation that includes a water-swellable polymer.

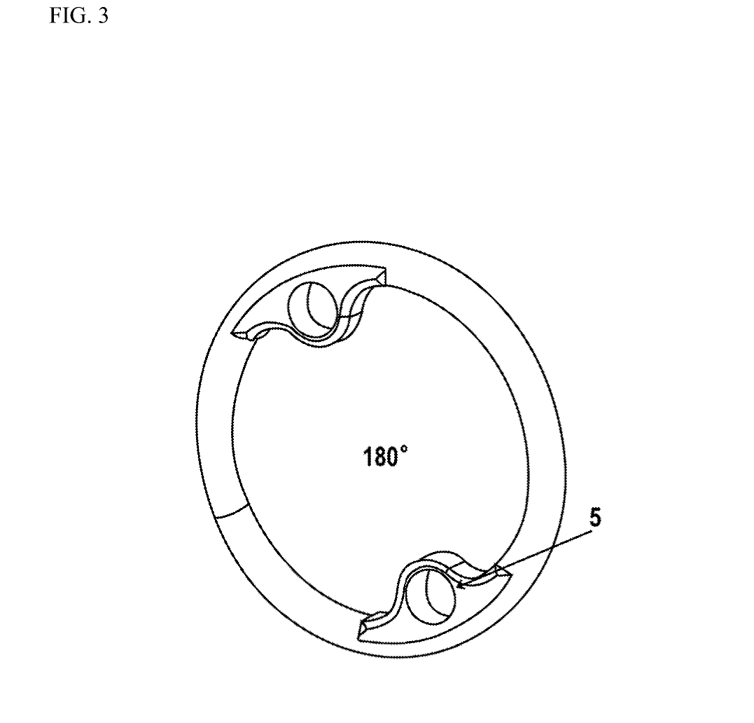

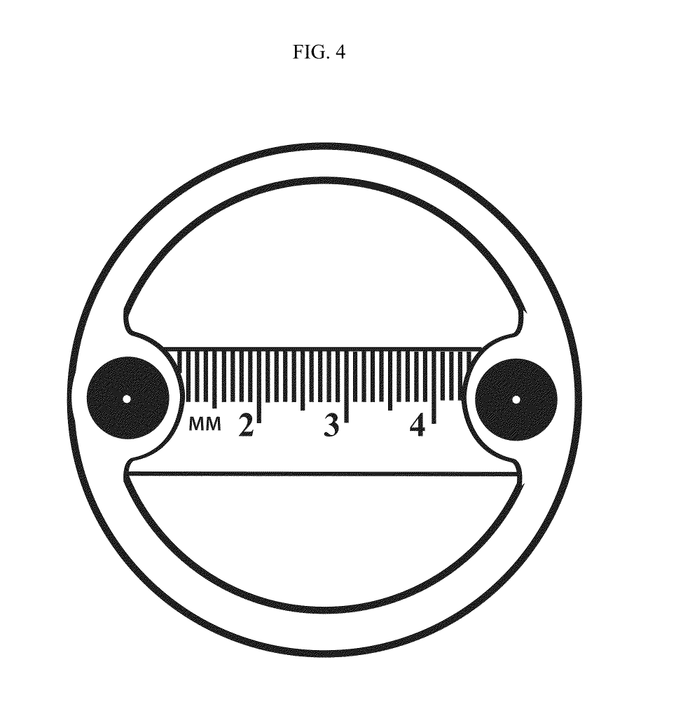

FIG. 3 shows a schematic drawing of an intravaginal ring with two bores designed to hold the pods. FIG. 4 shows a photograph of an intravaginal ring with pods, produced according to the procedure of this example.

Method 1C.

In an additional method, prototype pods and caps were fabricated using a lathe out of acrylonitrile butadiene styrene (ABS). The pellet was placed in the container and sealed with cap secured using an ABS adhesive. The completed pod is approximately 8.0 mm in diameter and 10-20 mm high with holes drilled on the side, and holds a compressed pellet of from 50-400 mg of a drug formulation that includes a water-swellable polymer.

### Example 2

### General Procedures for Quantification of Carrageenan and Other Compounds in Vaginal Fluid Simulant (VFS)

Carrageenans were quantified using a colorimetric assay based on methylene blue essentially as described by Soedjak (Anal. Chem. 1994, 66, 4514). Methylene blue interacts with carrageenans to form water-soluble metachromatic complexes at low concentrations of the reactants. The complexation results in a color change in the dye from blue to purple. The change in absorbance at 660 nm was used to determine concentration of carrageenan.

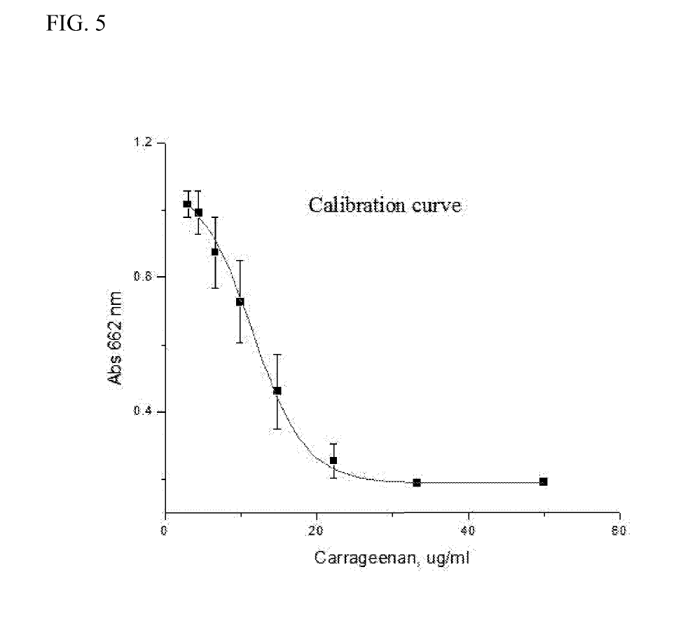

Calibration curves were generated for the range of carrageenan concentrations (3-50 μg/mL) in VFS. Briefly, a stock solution of carrageenan in VFS at concentration 50 μg/mL was prepared. Six serial dilutions with a step of 1.5 were done to obtain standard solutions with the following concentrations: 50 μg/mL, 33.3 μg/mL, 22.2 μg/mL, 14.8 μg/mL, 14.8 μg/mL, 9.8 μg/mL, 6.5 μg/mL. 100 μL of the standard solutions were added to wells of the 96 plate. 10 μL of solution of methylene blue in DDI water at concentration of 0.2 mg/mL were added to wells with a solution of carrageenan. The plate was placed into the plate reader and absorbance at 660 nm was measured. These measurements were used to generate calibration curves. OriginPro 8 software was used to perform fitting and analyze data. A typical calibration curve is shown in FIG. 5.

VFS was prepared according to the procedures in Owen D N, Katz D F Contraception 1999 February; 59(2):91-5. Specifically, 1 liter of simulant contained the following components (by weight in grams): NaCl, 3.51; KOH, 1.40; Ca(OH)₂, 0.222; bovine serum albumin, 0.018; lactic acid, 2.00; acetic acid, 1.00; glycerol, 0.16; urea, 0.4; glucose, 5.0.

### Example 3

### Release of Iota-Carrageenan from the Pod

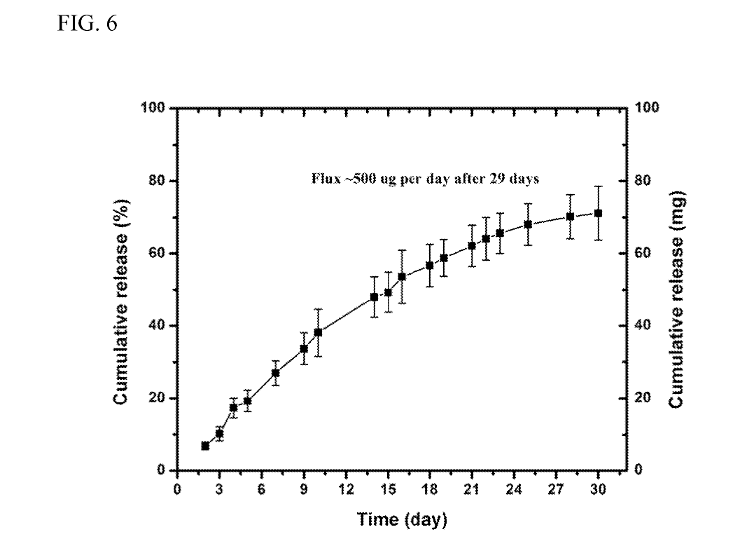

Iota-carrageenan type V (Sigma) and iota-carrageenan (Gelcarin PC379, FMS Biopolymer) in powder form were directly compressed at 2 T for 20 sec. Pellets from iota-carrageenan type V(Sigma) were mounted into the pod of Example 1 (1^{st} method) with a release orifice of 1.5 mm. Pellets from iota-carrageenan (Gelcarin PC379, FMS Biopolymer) were mounted into the pod of Example 1 (2^{nd} method) with a release orifice 1.5 mm. Release was evaluated in 10 mL of VSF at 37° C. upon shaking in rotary shaker at 90 rpm. At designated time intervals indicated in FIG. 6 all fluid was replaced with fresh fluid each day.

The release of iota carrageenan was quantified according to the procedure in Example 2. FIG. 6 shows the cumulative release profile of iota-carrageenan type V(Sigma) and FIG. 7 shows the cumulative release profile of iota-carrageenan (Gelcarin PC379, FMS Biopolymer).

### Example 4

### Effect of the Cationic Excipient Lysine on the Rate of Lambda-Carrageenan Release

Lysine (Sigma) in powder form was added to powdered lambda-carrageenan (Viscarin GP109 NF, FMC Biopolymer) at 20 wt % and 50 wt %. They were mixed in a mortar and compressed into pellets. The weights of the pellets were 120 mg for 20 wt % lysine and 150 mg for 50 wt % lysine, with 100 mg of carrageenan per pellet. Compression was done at 2 T for 20 sec. Pellets were mounted into the pod of Example 1 (1^{st} method) with a release orifice of 1.5 mm. Release was evaluated in 10 mL of VSF at 37° C. upon shaking in rotary shaker at 90 rpm. At designated time intervals indicated in FIG. 8, all fluid was replaced with fresh fluid. The release of carrageenan in the presence of lysine was quantified using the procedures of Example 2; cumulative release profiles are shown in FIG. 8.

### Example 5

### Release of BSA-FITC from an Iota-Carrageenan Matrix

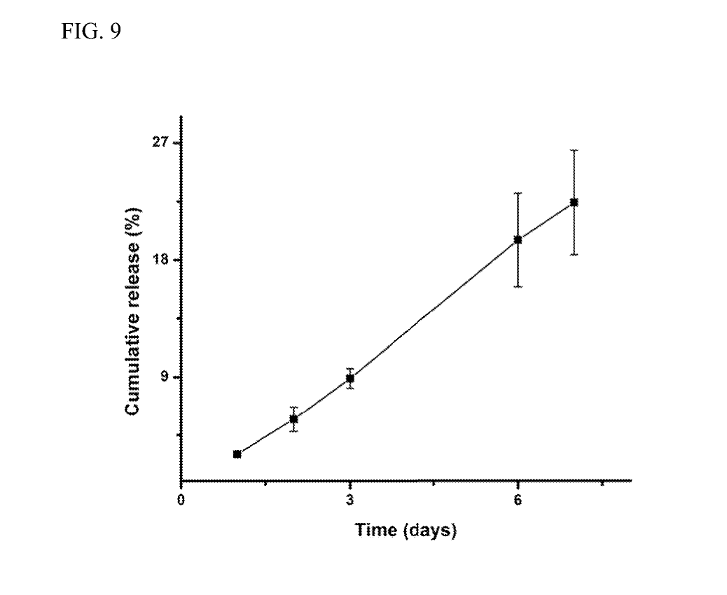

Fluorescein isothiocyanate (FITC) labeled bovine serum albumin (BSA) (1 mg) was mixed with 400 mg iota-carrageenan (Gelcarin PC 379, FMS Biopolymer Inc. in a mortar and pestle. Pellets were formed from 100 mg of the mixture by compression at 2 T for 20 sec. The pellets were mounted into the pod of Example 1 (1^{st} method) with a release orifice of 1.5 mm. Release was evaluated in 10 mL of VSF at 37° C. upon shaking in a rotary shaker at 90 rpm. At designated time intervals all fluid was replaced with fresh VFS. The release of carrageenan was quantified using the procedures of Example 2; the cumulative release profiles are shown in FIG. 9. The mixture (50 mg) was used to prepare a solution to generate a calibration curve for quantification of BSA-FITC in the release medium. Intensity of fluorescence at excitation of 488 nm and emission at 520 was used to determined concentration of BSA-FITC in solutions collected for analysis.

### Example 6

### Effect of Compression Force on Release of Iota-Carrageenan

Iota-carrageenan (Gelcarin PC379, FMS Biopolymer) were directly compressed at 0.5 T and 2 T for 20 sec. Pellets were mounted into the experimental pod (FIG. 1 showing its design) with a release orifice 1.5 mm. Release was evaluated in 10 mL of VSF at 37° C. upon shaking in a rotary shaker at 90 rpm. At designated time intervals all fluid was replaced with fresh VFS. The release of carrageenan was quantified using the procedures of Example 2; the cumulative release profiles are shown in FIG. 10.

### Example 7

### Effect of a Release Orifice Size on Rate of a Carrageenan Release

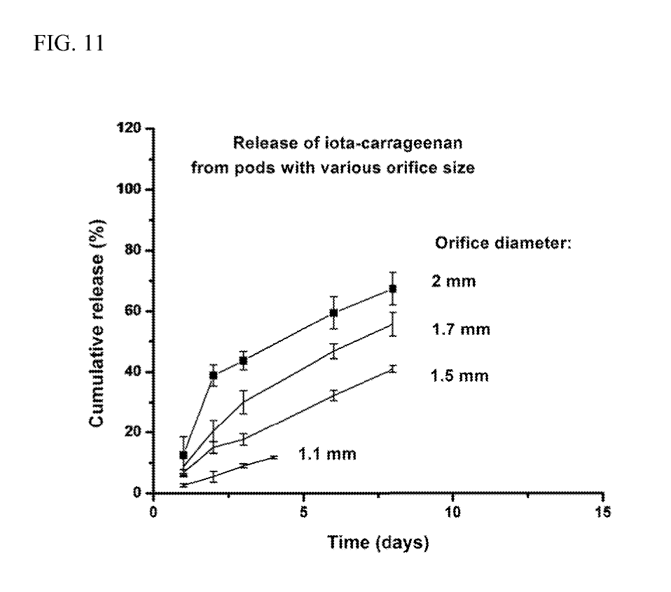

Iota-carrageenan (Gelcarin NF 379, FMS Biopolymer) (100 mg) was compressed at 2 T for 20 seconds into pellets. Pellets were mounted into the pods as in Example 1 (1^{st} method) with various release orifices (1.1, 1.5, 1.7, and 2 mm). Release was evaluated in 10 mL of VSF at 37° C. upon shaking in rotary shaker at 90 rpm. At designated time intervals all fluid was replaced with fresh VFS. The release of carrageenan was quantified using the procedures of Example 2; the cumulative release profile is shown in FIG. 11

### Example 8

### Effect of Intensity Swelling of Gel Matrix on Rate of Carrageenan Release

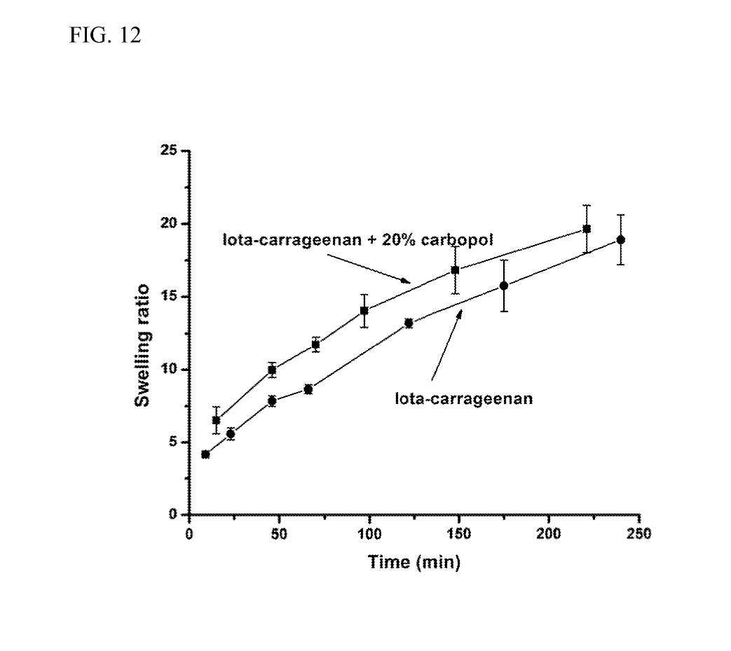

Iota-carrageenan (Gelcarin NF 379, FMS Biopolymer) and carbopol 974P NF (Noveon) 20 wt % of the total mass were wet granulated with doubly deionized water. Obtained mass was dried at 40 C for overnight. After milling by pestle and mortar 120 mg pellets were compressed at 2 T for 20 sec. Pellets were mounted into the experimental pod (FIG. 1 showing its design) with a release orifice 1.5 mm. Release was evaluated in 10 mL of VSF at 37° C. upon shaking in rotary shaker at 90 rpm. At designated time intervals all fluid was replaced with fresh VFS. The release of carrageenan was quantified using the procedures of Example 2; the cumulative release profile is shown in FIG. 12. The results show that adding carbopol to the carrageenan pellet results in higher swelling ratios of the pellet upon hydration.

### Example 9

### Release of Fluorescein as a Low Molecular Weight Model of Drug Release from an Iota-Carrageenan Matrix

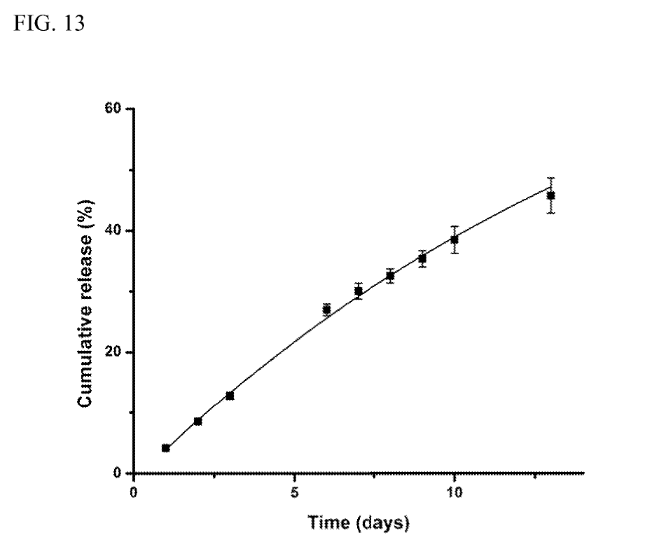

Fluorescein (1.5 wt %) was mixed with iota-carrageenan (Gelcarin PC 379, FMS Biopolymer Inc. in a mortar and pestle. Pellets were formed from 100 mg of the mixture by compression at 2 T for 20 sec. The pellets were mounted into the pod of Example 1 (1^{st} method) with a release orifice of 1.5 mm. Release was evaluated in 10 mL of VSF at 37° C. upon shaking in a rotary shaker at 90 rpm. At designated time intervals all fluid, as indicated on the plot showing fluorescein release (FIG. 13), was replaced with fresh VFS. The release of fluorescein was quantified according to a described below procedure. The mixture of fluorescein and carrageenan (50 mg) was used to prepare a solution to generate a calibration curve for quantification of fluorescein in the release medium. Intensity of fluorescence at an excitation of 488 nm and an emission at 520 was used to determine fluorescein concentration.

### Example 10

### Effect of Ethyl Cellulose on Release of Iota-Carrageenan

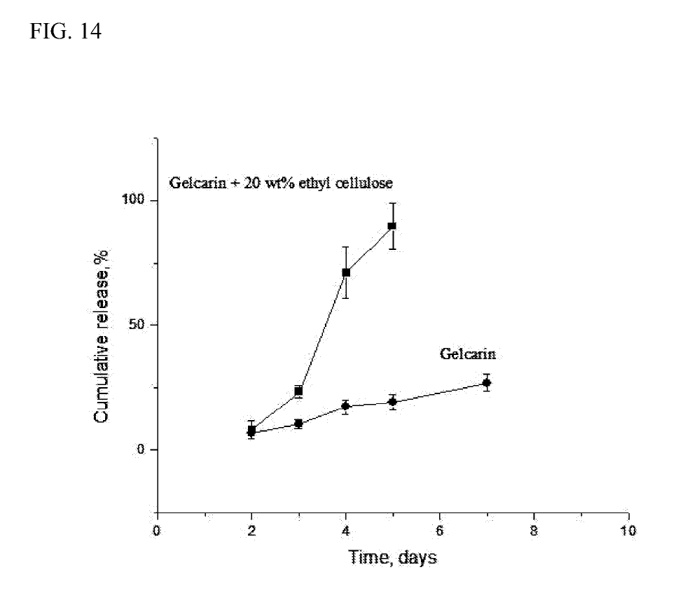

Iota-carrageenan (Gelcarin NF 379, FMS Biopolymer) and ethyl cellulose (Aldrich) 20 wt % of the total mass were wet granulated with ethanol. The resulting mixture was dried at 40° C. overnight. After milling by pestle and mortar, 120 mg pellets were compressed at 2 T for 20 sec. Pellets were mounted into the pod of Example 1 (1^{st} method) with a release orifice 1.5 mm. Release was evaluated in 10 mL of VSF at 37° C. upon shaking in rotary shaker at 90 rpm. At designated time intervals all fluid was replaced with fresh VFS. The release of carrageenan was quantified using the procedures of Example 2; the cumulative release profile is shown in FIG. 14

The results of Examples 3-10 are summarized in Table 1 as well as additional examples according to the same procedures.

TABLE 1

| | | | Orifice size | | POD |
|---|---|---|---|---|---|
| Formulation | Supplier | Pellet Design | (mm) | | Type* |
| Iota-CG | Gelcarin PC | 100 mg tablet, | 1.5 | 40% cumulative | 1 |
| | 379, FMS | 2 Ton | | release after 10 | |
| | Biopolymer | compression | | days | |
| Iota-CG + | Gelcarin PC | 100 mg tablet, | 1.5 | 22% cumulative | 1 |
| FITC-BSA | 379, FMS | 2 Ton | | release after 7 | |
| | Biopolymer | compression | | days | |
| Iota-CG | Gelcarin PC | 100 mg tablet, | 1.2 | 12% cumulative | 1 |
| | 379, FMS | 2 Ton | | release after 3 | |
| | Biopolymer | compression | | days | |
| Iota-CG | Gelcarin PC | 100 mg tablet, | 0.9 | 9% cumulative | 1 |
| | 379, FMS | 2 Ton | | release after 3 | |
| | Biopolymer | compression | | days | |
| Iota-CG | Gelcarin PC | 100 mg tablet, | 0.7 | 2% cumulative | 1 |
| | 379, FMS | 2 Ton | | release after 3 | |
| | Biopolymer | compression | | days | |
| Iota-CG | Gelcarin PC | 100 mg tablet, | 0.6 | 1.5% | 1 |
| | 379, FMS | 2 Ton | | cumulative | |
| | Biopolymer | compression | | release after 3 | |
| | | | | days | |
| 80% Iota-CG | Gelcarin PC | 100 mg tablet, | 1.2 | 1.5% 60% | 1 |
| and 20% | 379, FMS | 2 Ton | | cumulative CG | |
| Carbopol | Biopolymer, | compression | | release after 6 | |
| | Carbopol 949 P | | | days | |
| Iota-CG (11- | Type V | 100 mg tablet, | 1.5 | 70% cumulative | 1 |
| 18-09) | Sigma-Aldrich | 500 Kg/cm² | | release after 30 | |
| | | compression | | days | |
| Iota-CG | Gelcarin PC | 100 mg tablet, | 1.5 | 70% | 1 |
| | 379, FMS | 500 Kg/cm² | | cumulative | |
| | Biopolymer | compression | | release after 15 | |
| | | | | days | |
| Iota-CG + | Gelcarin PC | 100 mg tablet, | 1.5 | 60% | 1 |
| 10% ethyl | 379, FMS | 500 Kg/cm² | | cumulative | |
| cellulose | Biopolymer | compression | | release of i-CG | |
| | | | | after 15 days | |
| Iota-CG + | Gelcarin PC | 100 mg tablet, | 1.5 | 90% | 1 |
| 20% ethyl | 379, FMS | 500 Kg/cm² | | cumulative | |
| cellulose | Biopolymer | compression | | release of i-CG | |
| | | | | after 15 days | |
| Iota-CG + | Gelcarin PC | 100 mg tablet, | 1.5 | 65% | 1 |
| 20% Lysine | 379, FMS | 500 Kg/cm² | | cumulative | |
| | Biopolymer | compression | | release of i-CG | |
| | | | | after 10 days | |
| Iota-CG + | Gelcarin PC | 100 mg tablet, | 1.5 | 45% | 1 |
| 50% Lysine | 379, FMS | 500 Kg/cm² | | cumulative | |
| | Biopolymer | compression | | release of i-CG | |
| | | | | after 10 days | |
| iota-CG type V | (Sigma). | 100 mg tablet, | 1.5 mm | 70% | 1 |
| | | 500 Kg/cm² | | cumulative | |
| | | compression | | release of i-CG | |
| | | | | after 30 days | |

*1 = pod produced by Method 1A of Example 1; 2 = pod produced by the Method 1B of Example 1.

### Example #11

### Release of TAMRA-Insulin from Iota-Carrageenan, HPC and HEC Matrixes

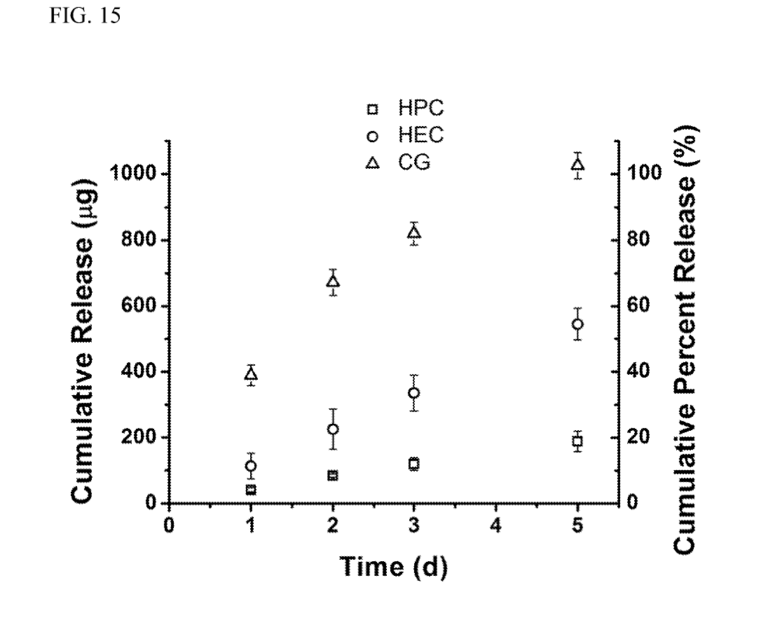

TAMRA-insulin (TAMRA, also TAMRA-SE, 5/6-carboxy-tetramethyl-rhodamine succinimidyl ester) was mixed with Iota-carrageenan (Gelcarin NF 379, FMS Biopolymer), hydroxyethyl cellulose (HEC, Natrusol 250 HX) and hydroxypropyl cellulose (Klucel HPC, GF Pharm) at 1 wt % with a TissueLyser (Qiagen). 100 mg pellets were made by direct compression at 2 T for 20 s. Pellets were mounted into the pods as in Example 1 (Method 1C) with two 1.5 mm diameter release orifices. The completed pod is approximately 5.0 mm in diameter and 12 mm high. Release was evaluated in 5 mL of 25 mM acetate buffer (pH 4.2) at 37° C. upon shaking in rotary shaker at 80 rpm. Release media was replaced daily. The TAMRA-insulin/swelling polymer mixture was used to prepare a solution to generate a calibration curve for quantification of TAMRA-insulin in the release media. Excitation and emission wavelengths of 540 nm 620 nm, respectively, were used to determine the concentration of TAMRA-insulin. The cumulative release profile is shown in FIG. 15.

### Example #12

### Effect of Swelling Polymer Molecular Weight on Release of TAMRA-Insulin from HPC Matrix

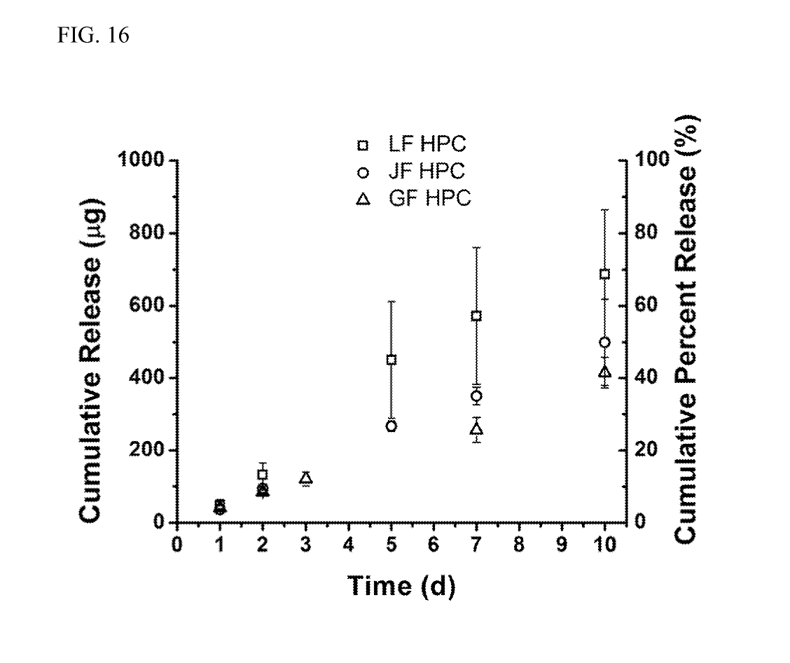

TAMRA-insulin was mixed for 3 minutes at 30 Hz with three different molecular weight HPCs (Klucel HPC; LF, JF and GF Pharm having weight average molecular weights of 95,000, 140,000, and 370,000, respectively) at 1 wt % using a TissueLyser (Qiagen). 100 mg pellets were made by direct compression at 2 T for 20 s. Pellets were mounted into the pods as in Example 1 (Method 1C) with two 1.5 mm release orifices. The completed pod is approximately 5.0 mm in diameter and 12 mm high. Release was evaluated in 5 mL of 25 mM acetate buffer (pH 4.2) at 37° C. upon shaking in rotary shaker at 80 rpm. Release media was replaced daily. The TAMRA-insulin/swelling polymer mixture was used to prepare a solution to generate a calibration curve for quantification of TAMRA-insulin in the release media. Excitation and emission wavelengths of 540 nm 620 nm, respectively, were used to determine the concentration of TAMRA-insulin. The cumulative release profile is shown in FIG. 16.

### Example #13

### Release of Rhodamine-B Dextran in a Rabbit Model

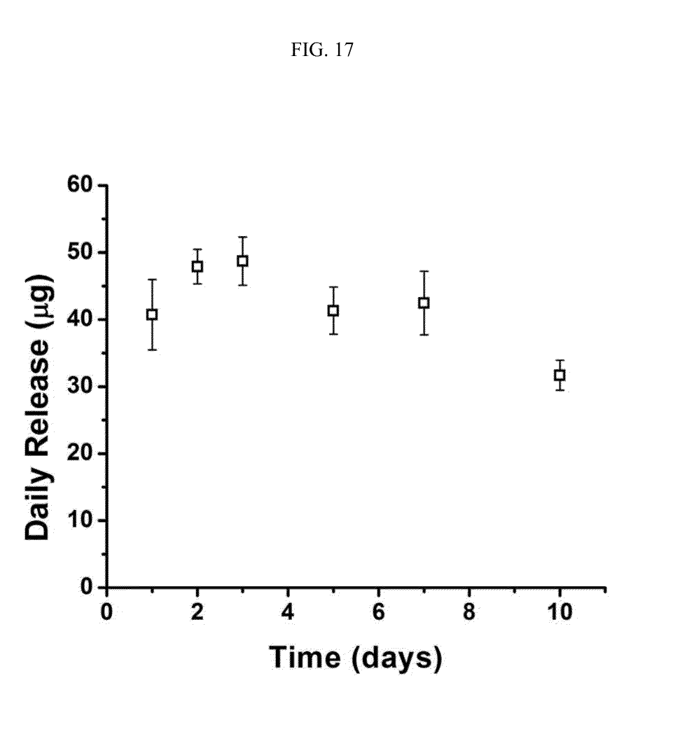

Rhodamine-B dextran (10 kDa) was mixed with HPC (Klucel HPC, GF Pharm) at 1.0 wt % with a TissueLyser (Qiagen). 100 mg pellets were made by direct compression at 2 T for 20 s. Pellets were mounted into the pods as in Example 1 (Method 1C) with two 1.5 mm release orifices. The completed pod is approximately 5.0 mm in diameter and 12 mm high. In vitro release was evaluated in 5 mL of 25 mM acetate buffer (pH 4.2) and phosphate buffered saline (PBS) (pH 7.4) at 37° C. upon shaking in rotary shaker at 80 rpm. In vitro release media was replaced daily. In vivo release was evaluated in a rabbit vaginal model. The rhodamine-B dextran/swelling polymer mixture was used to prepare a solution to generate a calibration curve for quantification of rhodamine-B dextran in the release media. Excitation and emission wavelengths of 540 nm 620 nm, respectively, were used to determine the concentration of rhodamine-B dextran. The pellet mass after release was measured by cutting open the pod and extracting the remainder pellet with water and drying the solution on a lyophilizer. The daily in vitro release is shown in FIG. 17. The pellet mass loss after 10 days was 48.2±1.9 mg, in vitro and 31.0±0.1 mg, in vivo.

TABLE 2
Summarized results from examples 11-13.

| | | | Orifice size | | POD |
|---|---|---|---|---|---|
| Formulation | Supplier | Pellet Design | (mm) | | Type* |
| Iota-CG + | Gelcarin PC | 100 mg tablet, | 1.5 | 100% | 3 |
| TAMRA- | 379, FMS | 2 Ton | | cumulative | |
| insulin | Biopolymer | compression | | release after 5 | |
| | | | | days | |
| HEC + FITC- | HEC, | 100 mg tablet, | 1.5 | 50% | 3 |
| dextran | Natrusol 250 | 2 Ton | | cumulative | |
| | HX | compression | | release after 5 | |
| | | | | days | |
| HPC + | Klucel HPC, | 100 mg tablet, | 1.5 | 20% | 3 |
| TAMRA- | GF Pharm | 2 Ton | | cumulative | |
| insulin | | compression | | release after 5 | |
| | | | | days | |
| HPC + | Klucel HPC, | 100 mg tablet, | 1.5 | 70% | 3 |
| TAMRA- | LF Pharm | 2 Ton | | cumulative | |
| insulin | | compression | | release after 10 | |
| | | | | days | |
| HPC + | Klucel HPC, | 100 mg tablet, | 1.5 | 50% | 3 |
| TAMRA- | JF Pharm | 2 Ton | | cumulative | |
| insulin | | compression | | release after 10 | |
| | | | | days | |
| HPC + | Klucel HPC, | 100 mg tablet, | 1.5 | 40% | 3 |
| TAMRA- | GF Pharm | 2 Ton | | cumulative | |
| insulin | | compression | | release after 10 | |
| | | | | days | |

*3 = pod produced by Method 1C of Example 1

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An intravaginal device comprising a chamber having at least one orifice and containing a sustained-release formulation, wherein the formulation comprises a water-swellable polymer and an intravaginally administrable substance.

2. The device of claim 1 wherein the water-swellable polymer is carrageenan, polyacrylic acid or hydroxyl ethylcellulose.

3. The device of claim 2 wherein the water-swellable polymer is lambda carrageenan, iota carrageenan, kappa carrageenan or a mixture of any two or more thereof.

4. The device of claim 1 wherein the chamber has a single orifice with a diameter of about 0.1 mm to about 4 mm.

5. The device of claim 1 comprising two to six chambers, each comprising at least one orifice and each containing a sustained-release formulation.

6. The device of claim 1 comprising two or three chambers, each comprising an orifice and containing a sustained-release formulations.

7. The device of claim 1 wherein the water-swellable polymer and the intravaginally administrable substance are the same.

8. The device of claim 1 wherein the device is an intravaginal ring, tampon or pessary.

9. The device of claim 8 wherein the device is an intravaginal ring formed from polyurethane, ethylene vinyl acetate, silicone or a mixture of any two or more thereof.

10. The device of claim 9 further comprising a second intravaginally administrable substance different from the first.

11. The device of claim 10 wherein the second intravaginally administrable substance is loaded into the ring itself or a second chamber having at least one orifice and containing a sustained-release formulation comprising a second water swellable polymer.

12. The device of claim 10 wherein the intravaginally administrable substance is a sulfated polysaccharide and the second intravaginally administrable substance is selected from an anti-viral or a contraceptive.

13. The device of claim 12 wherein the intravaginally administrable substance is carrageenan, and the second intravaginally administrable substance is a contraceptive loaded into one or more segments of the intravaginal ring which are surrounded by a sheath allowing for controlled release of the contraceptive.

14. The device of claim 2 wherein the sustained release formulation further comprises an excipient selected from the group consisting of cellulose, xanthan gum, amino acids, and glucosamine.

15. The device of claim 14 wherein the excipient is selected from the group consisting of lysine, histidine, or arginine.

16. The device of claim 14 wherein the excipient is ethyl cellulose.

17. The device of claim 1 wherein release of the water-swellable polymer in intravaginal simulant fluid continues for at least 7 days, at least 14 days, or at least 30 days.

18. The device of claim 1, wherein the intravaginally administrable substance is selected from the group consisting of cervical anesthetics, contraceptives, antiendometriosis drugs, estrogen receptor modulators, preterm labor drugs, overactive bladder drugs, morning sickness drugs, osteoporosis drugs, drugs and/or substances for vaginal dryness and antimicrobials.

19. The device of claim 18 wherein the intravaginally administrable substance is a drug selected from the group consisting of etonogestrel and ethinyl estradiol, and a combination thereof.

20. The device of claim 18 wherein the intravaginally administrable substance is levonogestrel

21. The device of claim 18, wherein the substance is an antiviral, antifungal or antibacterial.

22. The device of claim 21, wherein the substance is selected from the group consisting of lambda carrageenan, iota carrageenan, kappa carrageenan, a polypyrrole and a combination of any two or more thereof.

23. The device of claim 18, wherein the substance is an anti-HIV agent selected from the group consisting of non-nucleoside reverse transcriptase inhibitors, nucleoside reverse transcriptase inhibitors, and HIV entry inhibitors.

24. The device of claim 23, wherein the non-nucleoside reverse transcriptase inhibitor is dapivirine and the nucleoside reverse transcriptase inhibitor is Tenofovir.

25. The device of claim 18, wherein the substance is selected from 1-(cyclopent-3-enylmethyl)-6-(3,5-dimethylbenzoyl)-5-ethylpyrimidine-2,4(1H,3H)-dione, 1-(cyclopentenylmethyl)-6-(3,5-dimethylbenzoyl)-5-isopropylpyrimidine-2,4(1H,3H)-dione, 1-(cyclopent-3-enylmethyl)-6-(3,5-dimethylbenzoyl)-5-isopropylpyrimidine-2,4(1H,3H)-dione, 1-(cyclopropylmethyl)-6-(3,5-dimethylbenzoyl)-5-isopropylpyrimidine-2,4(1H,3H)-dione, 1-(4-benzoyl-2,2-dimethylpiperazin-1-yl)-2-(3H-pyrrolo[2,3-b]pyridin-3-yl)ethane-1,2-dione, or 19-norethindrone.

26. A method comprising loading a sustained-release formulation into a chamber of an intravaginal device wherein the chamber has at least one orifice and wherein the sustained-release formulation comprises a water-swellable polymer and an intravaginally administrable substance.

27. The method of claim 26 further comprising compressing particles of the water-swellable polymer and the substance, optionally mixed with one or more excipients, into a pellet before loading the pellet into the chamber of the intravaginal device.

28. The method of claim 27 wherein the particles have been put through a sieve having a mesh size of from about 200 μM to about 80 μM prior to compression.

29. The method of claim 27 wherein the particles were compressed at a pressure of about 0.5 T to about 10 T.

30. The method of claim 26, wherein the water-swellable polymer is carrageenan.

31. A method comprising releasing a substance from an intravaginal device comprising a chamber having at least one orifice and containing a sustained-release formulation, wherein the formulation comprises a water-swellable polymer and a intravaginally administrable substance.
